(19) United States
(12) Patent Application Publication
(10) Pub. No.: US 2003/0114925 A1
(43) Pub. Date: Jun. 19, 2003

(54) Graphite reinforced pyrolytic carbon heart valve prosthesis and method

(76) Inventor: Gerardo Molina, Leander, TX (US)

Correspondence Address: Timothy L. Scott, Senior, Intellectual Property Counsel, SULZER MEDICA USA INC., 3 East Greenway Plaza, Suite 1600, Houston, TX, 77046, US

(21) Appl. No.: 10/022,887

(22) Filed: Dec. 18, 2001

(51) Int. Cl. A61F 2/24

(52) U.S. Cl. 623/2.38; 623/2.42; 427/2.24

(57) ABSTRACT

A mechanical prosthetic heart valve with a pyrolytic carbon valve body having re-enforcing fibers in the valve body. The fibers may be short segments of graphite wire or continuous fibers may be wrapped around the valve body. The fiber may be arranged in loops and formed in repeating patterns around the valve body. Also, methods for making a prosthetic heart valve body having a first layer of pyrolytic carbon that is substantially free from fiber, a second layer comprised of fibers encased in pyrolytic carbon, and a third layer that is substantially free from fiber. The method may comprise placing a mandrel in a fluidized bed, coating the mandrel with a first layer of pyrolytic carbon, introducing a fiber, coating the mandrel and the fiber with a second layer of pyrolytic carbon, and coating the mandrel with a third layer of pyrolytic carbon.

### TECHNICAL FIELD

The present invention pertains to prosthetic heart valves and in particular to pyrolytic carbon heart valve prostheses.

### BACKGROUND ART

Ever since 1950, when blood oxygenators made open heart surgery feasible, it has been possible to treat some forms of heart disease by replacing one of the patient's heart valves with a mechanical heart valve. Typically, a mechanical heart valve comprises an annular valve body in which two opposed leaflet occluders are pivotally mounted. The occluders are typically substantially rigid, although some designs incorporate flexible leaflets, and move between a closed position, in which the two leaflets mate and block blood flow in the reverse direction, and an open position, in which the occluders pivot away from each other and allow blood to flow in the forward direction. The energy of blood flow causes the occluders to move between their open and closed positions. Typically the valve body and the occluders comprise structures of pyrolytic carbon, a durable, bio-compatible material.

One of the issues considered in the design of mechanical heart valves is the strength and associated thickness of the annular valve body. Pyrolytic carbon is a hard but relatively brittle material. To achieve the required strength in compression, a metal stiffening ring often circumscribes the annular valve body. The stiffening ring also connects a sewing ring to the annular valve body. The size of the implant site for a valve in a particular patient controls the outer diameter of a heart valve that can be implanted at that site. With the outer diameter of the prosthesis determined by the physiology of the patient, the combined thickness of the sewing ring, stiffening ring and annular valve body limits the size of the central orifice through which blood flows. It is desirable to have as large a central orifice as possible, since larger orifices present less resistance to blood flow and reduce the work done by the heart in pumping blood through the body. It would be desirable, therefore, to reduce or eliminate the stiffening ring and minimize the thickness of the annular valve body.

It is an object of the present invention, therefore, to provide a mechanical heart valve with a strengthened pyrolytic carbon valve body.

It is further an object of the invention to provide a pyrolytic carbon valve body with improved strength and resistance to compression or to cracking.

Another object of the invention is to provide a mechanical heart valve that does not require an additional stiffening ring.

A further object of the invention is to provide a manufacturing process for pyrolytic carbon valve bodies.

These and other objects and features of the invention will be apparent from the following description.

### BRIEF SUMMARY OF THE INVENTION

In one aspect of the invention, a mechanical heart valve with a pyrolytic carbon valve body has re-enforcing fibers in the valve body. The fibers may be short segments of graphite wire having a critical length such that the valve body is strengthened to a similar degree as if continuous fiber had been wrapped around the valve body. In another aspect of the invention, one or more continuous fibers may be wrapped around the valve body. The fiber may be arranged in loops, each loop lying adjacent another loop. The loops may also be spaced apart from each other. The fiber may also be formed in generally sinusoidal or other repeating pattern around the valve body.

Preferably, the annular valve body defines an orifice through which blood flows and has a first layer adjacent the orifice that is substantially free from fibers. The first layer comprises mainly pyrolytic carbon and presents a smooth, uniform, bio-compatible surface to the flow of blood through the orifice. A second layer, circumferentially surrounding the first layer, preferably comprises fiber segments or wire encased in more pyrolytic carbon. A third layer, circumferentially surrounding the second layer, has a different composition from the second layer, in that the third layer is substantially free from fibers. The second layer, therefore, is sandwiched between the first and third layers. Few or no fibers are exposed to blood or body fluids.

The fiber-filled second layer increases the toughness of the valve body, making the valve body more resistant to cracking. Increased toughness permits mechanical heart valve designs with thinner sidewalls, whereby the central orifice may be made larger. A larger orifice generally has less resistance to blood flow. The valve body may be made sufficiently tough such that a metal stiffening ring may be omitted or at least substantially reduced in size. The reduction or omission of the stiffening ring also means that the heart valve can have a larger central orifice for a selected outside diameter.

The invention also comprises methods for making a mechanical heart valve or valve body wherein the valve body has a first inside layer of pyrolytic carbon that is substantially free from fiber, a second inner layer comprised of fibers encased in pyrolytic carbon, and a third outside layer that is substantially free from fiber. The method may comprise placing a mandrel in a fluidized bed, coating the mandrel with a first layer of pyrolytic carbon, introducing a fiber, coating the mandrel and the fiber with a second layer of pyrolytic carbon, and coating the mandrel with a third, substantially fiber-free layer of pyrolytic carbon. The step of introducing a fiber may include inserting chopped or short fibers into the fluidized bed. The short fibers may be equal to or greater than a critical length, that is, long enough that the increased strength of the composite structure is similar to that of a composite structure having one or more continuous fibers wrapped circumferentially around the mandrel and first layer. The step of introducing a fiber may also include wrapping coils of a fiber around the first coating on the mandrel. The wrapped coils may form a pattern such as a sinusoidal pattern or other repeating pattern. Layers of fibers may be laid one above the other. The fiber may be graphite fiber.

### BRIEF DESCRIPTION OF THE DRAWINGS

FIG. 1 is a perspective view of a mechanical prosthetic heart valve.

FIG. 2 is a cross sectional view of a fluidized bed coating apparatus.

FIG. 3 is a cross sectional view of a first layer of an annular valve body on a mandrel.

FIG. 4 is a cross sectional view of the annular valve body of FIG. 3 with a second layer having implanted fibers.

FIG. 5 is a cross sectional view of the annular valve body of FIG. 4 with a third layer.

FIG. 6 is a cross sectional view of the annular valve body of FIG. 5 after removal of the mandrel and machining of outside surfaces.

FIG. 7 is a perspective view of a first layer of an annular valve body on a mandrel illustrating circumferential fibers.

FIG. 8 is a cross sectional view of the valve body of FIG. 7.

FIG. 9 is a perspective view of the partially assembled annular valve body illustrating another pattern of wrapped fibers.

### DETAILED DESCRIPTION OF THE PREFERRED EMBODIMENT

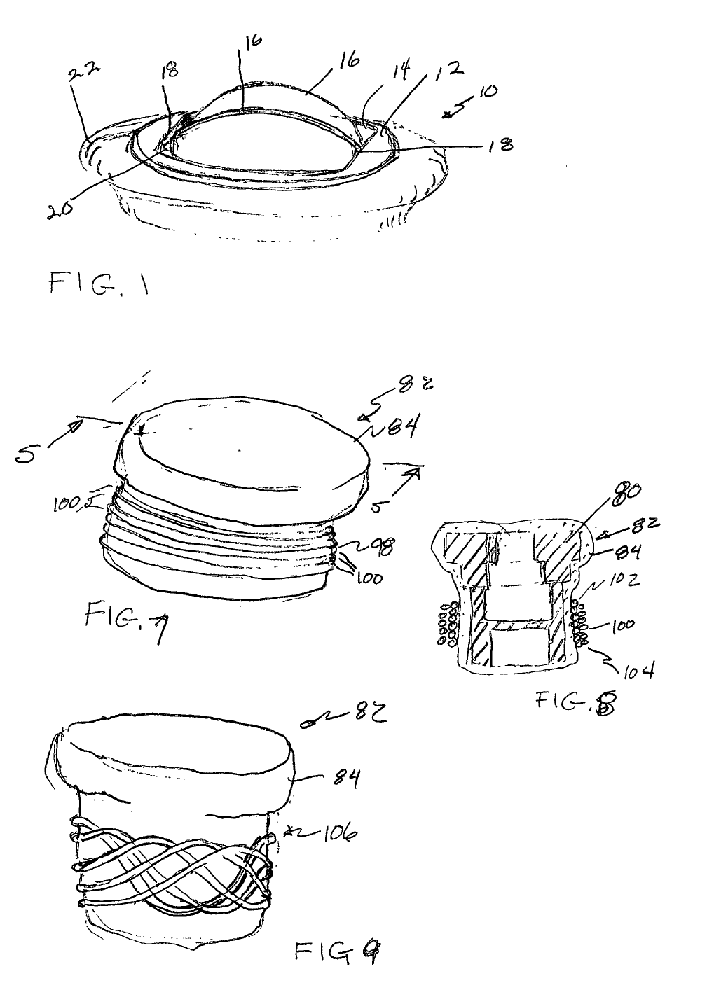

A mechanical heart valve 10, as illustrated in FIG. 1, usually has an annular valve body 12. The valve body 12 is formed of pyrolytic carbon. According to the invention, the pyrolytic carbon valve body has imbedded fibers which strengthen the valve body. The valve body 12 has an inner wall 14 that is substantially circular, but interrupted by two opposed, parallel flats 18. Two occluders 16 are mounted between the flats and pivot around pins or ears (not shown) that are mounted in recesses 20. The occluders 16 move between an open position, as shown, and a closed position, alternately permitting and resisting the flow of blood through the valve. A fabric sewing ring 22 circumferentially surrounds the valve body 12. The sewing ring 22 and the valve body 12 are connected, usually by a stiffening ring (not shown), as is well known in the art. The annular valve body may be sufficiently strengthened by the implanted fibers such that the stiffening ring may be made very thin or omitted entirely. The additional strength and toughness of the valve body allows a larger orifice or central opening in the valve for a given outside diameter of the valve. Since resistance to blood flow is inversely related to orifice size, a heart valve with a larger orifice has less resistance to blood flow. Minimizing resistance to blood flow is an important goal of mechanical heart valve design.

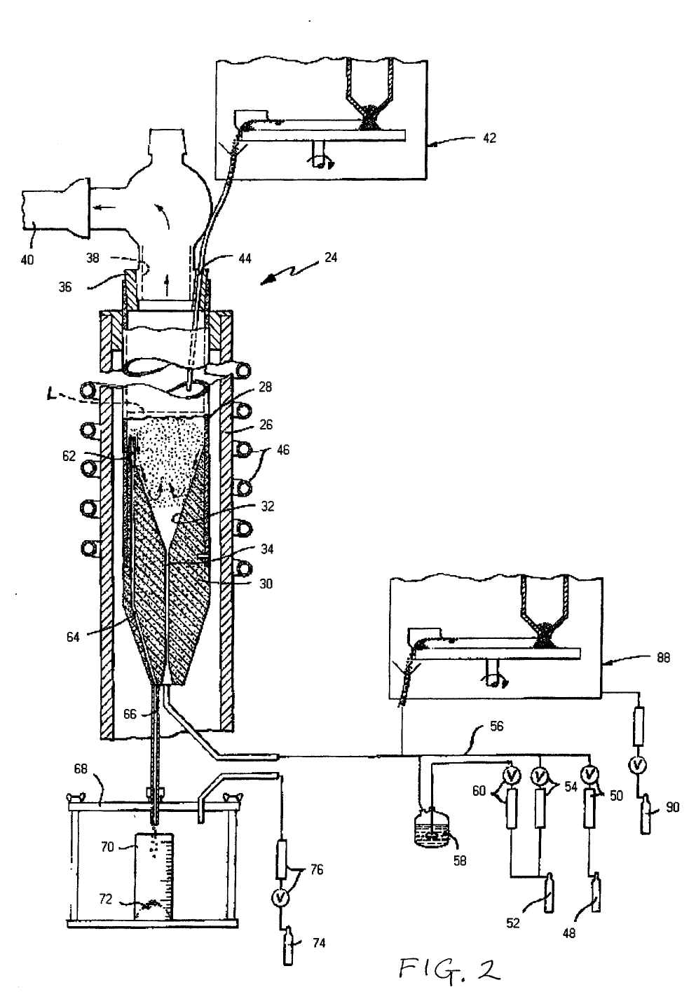

The annular valve body is made by depositing pyrolytic carbon over a mandrel suspended in a fluidized bed. A suitable fluidized bed coating apparatus 24 is depicted in the FIG. 2. The apparatus 24 includes a furnace having cylindrical outer shell 26. The shell 26 supports the coating enclosure which is defined by a tube 28 having an insert 30 affixed thereto at its lower end. The insert 30 provides the internal coating enclosure with a conical bottom surface 32. A central passageway 34 extends vertically upward through the insert 30, coaxial with the tube 28, and the coating and fluidizing atmosphere is supplied upward through this passageway.

The upper end of the tube 28 is provided with a removable closure 36 that may be mounted in any suitable manner. The closure 36 includes a central exit passageway 38 through which the fluidizing and coating gases leave the furnace enclosure and which is connected to an exit conduit 40. An injection device 42 is mounted above the closure 36 and is designed to feed fluidizing beads into the coating enclosure at a desired rate by dropping them downward through an opening 44 in the closure. The objects being coated may also be introduced through opening 44 in the closure. The beads will fall nearly the length of the tube 28 until they enter and become part of the fluidized bed. Induction heaters 46, which are well known in this art, are provided outside the lower end of the shell 26. The induction heaters 46 heat the active deposition region of the furnace and the beads and objects being coated to the desired deposition temperature.

In the fluidized bed coating apparatus 24, sometimes called a “steady-state bed”, the fluidizing beads and objects being coated are levitated near the bottom of the heating enclosure by the upward flowing gas stream. The gas stream is usually made up of a mixture of an inert fluidizing gas plus a gaseous hydrocarbon, for example, methane, ethane, propane, butane, or acetylene, or some other carbon-containing substance that is gaseous or easily vaporizable. In FIG. 2, a source 48 of hydrocarbon is illustrated which is equipped with a flow-regulating valve arrangement 50. Also illustrated is a source 52 of inert gas, for example, helium, argon or nitrogen, which is likewise equipped with a suitable flow-regulating valve arrangement 54. These two sources flow into a common line 56 which connects to the vertical passageway 34 in the insert 30.

Instead of depositing a coating which is entirely pyrolytic carbon, suitable carbide-forming additives have been co-deposited along with the pyrolytic carbon. For example, silicon, which forms silicon carbide, can be dispersed as silicon carbide throughout the pyrolytic carbon in an amount up to about 20 weight percent based on the total weight of the coating and will add strength to the pyrolytic carbon structure without detracting from otherwise desirable physical properties of the pyrolytic carbon. Examples of other carbide-forming elements which might be used as additives include boron, tungsten, tantalum, niobium, vanadium, molybdenum, aluminum, zirconium, titanium and hafnium. Usually, the carbide-forming additive is incorporated within the fluidizing and coating atmosphere by bubbling all or a part of the inert gas stream through a bath 58 containing a volatile liquid compound of the additive element in question. As illustrated in FIG. 2, a suitable flow control valve arrangement 60 is provided to regulate the proportion of the inert gas that will be passed through the additive bath 58.

A cross-section view of a mandrel 80 with annular valve body 82 is illustrated in FIG. 3. The mandrel 80 will be suspended in the fluidized bed as the object being coated. The fluidized bed deposits a first layer 84 of pyrolytic carbon on the mandrel 80. The first layer 84 may have molecular additives as described above, but is preferably substantially uniform in composition, texture, and other properties. The first layer reaches a selected thickness, as a function of the length of time the mandrel 80 has been suspended in the fluidized bed. Fiber segments 86 are then injected into the common line 56 of the upward flowing gas stream. Preferably the fiber segments are graphite fibers. The mixture of inert fluidizing gas plus a gaseous hydrocarbon carries the fiber segments 86 through the common line 56 and the vertical passageway 34 in the insert 30. A dispenser 88 delivers fiber segments 86 at a selected rate. The dispenser 88 may have a source 90 of inert gas that pressurizes the dispenser and prevents the gas stream from flowing into the dispenser rather than into the fluidized bed.

The length of the fiber segments 86 may as long as or longer than a critical length, that is, a length which produces equivalent characteristics to a fiber wrapped continuously around a cylindrical object. In narrow structures, 1 mm to about 5 mm fibers embedded in a matrix can give substantially equivalent performance to continuous fibers embedded in the matrix. The annular valve body of a mechanical heart valve is narrow in radial thickness, such that randomly arranged fiber segments can provide increased toughness and resistance to cracking. A critical fiber length independent of applied stress has been defined as the minimum fiber length in which the maximum allowable fiber stress σ_{f} can be achieved. Where L_{c} is the critical length, d is the fiber diameter and τₘₐₓ is the maximum yield stress in shear for the matrix, the critical length is

$${L}_{c} = \frac{d * {σ}_{f}}{2 {τ}_{max}}$$

The maximum yield stress in shear τₘₐₓ is related to the tensile strength σₘ by the equation:

τₘₐₓ=½σₘ.

The critical fiber length estimates the limit of the narrowest possible structure in which implanted fibers will reach their ultimate stress. The behavior of short-fiber reinforced composite will approach the behavior of continuous-fiber reinforced composites when the fiber length is approximately fifty (50) times greater than the load-transfer length, that is, fifty (50) times greater than the critical length L_{c}. See e.g. Pilling, J. E. “Aligned Short Fibres,” course handout, class 8 of Mechanics of Composites, available at http://callisto.my.mtu.edu/MY472/mech/class8.html, or other references known in the art.

The fiber segments mix in the fluidized bed and deposit on the outer surface of the first layer 84 of the annual valve body. The fiber segments and pyrolytic carbon form a second layer 92 comprised of fiber segments imbedded in pyrolytic carbon, as illustrated in FIG. 4. The fiber segments are relatively heavy and elongated as compared to the fluidizing beads comprising the fluidized bed. Therefore, the fiber segments tend to move more slowly and to have a relatively greater surface area as compared to the beads. Consequently, the fiber segments more frequently attach to the carbon layer on the mandrel, as they are less likely to rebound off the mandrel than the beads. Once an initial attachment point has been made, a fiber will gradually become completely attached to the carbon layer on the mandrel by further deposition of pyrolytic carbon. After a selected period of time, injection of additional fiber segments is stopped. Fiber segments continue to adhere to the annular valve body and mandrel for a time, but the fibers are gradually coated with pyrolytic carbon and their density decreases, in a manner similar to the beads forming the fluidized bed. Fibers not adhered to the annular valve body move to the top of the fluidized bed, away from the suspended mandrel. After the fiber segments move toward the top of the fluidized bed, a third layer 94 deposits on the annular valve body. The third layer 94 preferably has a composition similar to the first layer 84, with uniform chemical composition and properties. Different additives may be added to or omitted from the gas flow, however, to provide desirable characteristics. After the third layer 94 reaches an appropriate thickness, the mandrel and annular valve body can be removed from the fluidized bed. Removing the mandrel from inside the annular valve body uncovers the inner surface 14, opposed flat surfaces 18 and recesses 20. An outside surface 96, illustrated in FIG. 6, is finished in a conventional manner, for example by milling and polishing. Leaflets and sewing ring are then mounted on the annular valve body, producing a mechanical heart valve, as shown in FIG. 1.

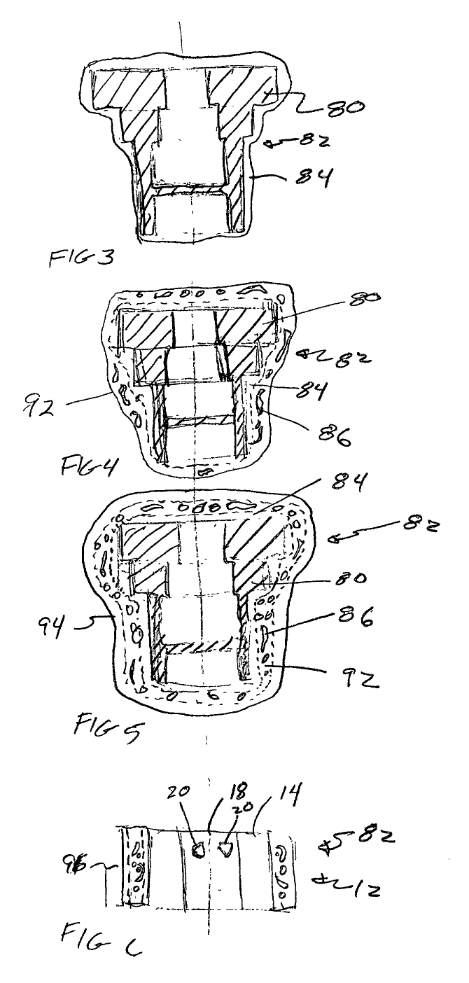

A fiber-reinforced annular valve body can also be constructed with continuous fibers. In this case, the mandrel is suspended in the fluidized bed until the first layer 84 has been deposited. Graphite fibers 98 are wound circumferentially around the first layer, as shown in FIG. 7, forming a plurality of coils 100. The adjacent coils may be in contact with each other, as in FIG. 7, or spaced apart. One or more layers 102, 104 may be provided, as shown in FIG. 8. After the fiber has been wound on the first layer of the annular valve body, the coated mandrel is returned to the fluidized bed. Continued deposition of pyrolytic carbon forms a second layer of fiber encased in pyrolytic carbon (see FIG. 4). The process is continued further until a third layer, substantially free from fiber, has been formed (see FIG. 5). The mandrel and valve body are then removed from the fluidized bed, and the mandrel is removed from the valve body, as described above. The valve body is milled, polished and supplied with leaflets and a sewing ring.

In addition to circumferential coils, other suitable patterns may be used to wrap continuous fiber around the first layer of the valve body. For example, a fiber 106 may be wrapped around the first layer in a regular pattern of offset sinusoidal waves 108, as shown in FIG. 9. Other patterns will suggest themselves to those skilled in the art in view of the present disclosure. As described above, the fiber pattern is then covered with pyrolytic carbon to form a composite layer. Further deposition produces the third layer, which is substantially free from fiber,

The foregoing describes preferred embodiments of the invention and is given by way of example only. The invention is not limited to any of the specific features described herein, but includes all variations thereof within the scope of the appended claims.

What is claimed is:

1. A method of manufacturing a pyrolytic carbon annular valve body for a mechanical heart valve comprising
forming a mandrel,
placing the mandrel in a fluidized bed,
coating the mandrel with a first layer of pyrolytic carbon,
introducing a fiber,
coating the mandrel with a second layer of pyrolytic carbon, said second layer including said fiber,
coating the mandrel with a third layer of pyrolytic carbon, said third layer not including fiber,
removing said mandrel from said coatings, and
shaping an outer surface of said coatings to form said annular valve body.

2. The method of claim 1 wherein introducing a fiber comprises inserting a plurality of fiber segments into said fluidized bed.

3. The method of claim 2 wherein said chopped fibers are of critical length.

4. The method of claim 1 wherein introducing a fiber comprises wrapping at least one fiber around said first layer.

5. The method of claim 4 further comprising removing said mandrel from said fluidized bed, wrapping said at least one fiber around said first layer and replacing said mandrel in said fluidized bed.

6. The method of claim 1 wherein said fiber is graphite wire.

7. An annular valve body for a mechanical heart valve comprising
an annular body defining a central orifice, said annular body having
a first circumferential layer of pyrolytic carbon, said first layer not including fiber,
a second circumferential layer of pyrolytic carbon, said second layer including fiber, and
a third layer of pyrolytic carbon, said third layer not including fiber.

8. The valve body of claim 7 wherein said fiber comprises a plurality of segments.

9. The valve body of claim 8 wherein said fiber segments are of critical length.

10. The valve body of claim 7 wherein said fiber comprises at least one fiber wrapped around said first layer to form at least one layer of fiber circumferentially around said first layer.

11. The valve body of claim 19 wherein said fiber is graphite wire.

12. A mechanical heart valve comprising
a pyrolytic carbon annular valve body defining a central orifice, said annular valve body having
a first circumferential layer of pyrolytic carbon, said first layer not including fiber,
a second circumferential layer of pyrolytic carbon, said second layer including fiber, and
a third layer of pyrolytic carbon, said third layer not including fiber, at least one leaflet pivotally received in said orifice, and
a sewing ring circumferentially mounted adjacent said third layer of pyrolytic carbon.

13. The mechanical heart valve of claim 12 wherein said fiber comprises a plurality of fiber segments.

14. The valve body of claim 13 wherein said fiber segments are of critical length.

15. The mechanical heart valve of claim 12 wherein said fiber comprises at least one fiber wrapped around said first layer to form at least one layer of fiber circumferentially around said first layer.

16. The mechanical heart valve of claim 12 wherein said fiber is graphite wire.
